(19) United States
(12) Patent Application Publication
(10) Pub. No.: US 2017/0073662 A1
(43) Pub. Date: Mar. 16, 2017

(54) MOLECULE CARRIER AND METHOD FOR PREPARING THE SAME

(71) Applicant: National Central University, Jhongli City (TW)

(72) Inventors: Fa-Kuen SHIEH, Pingzhen City (TW); Shao-Chun WANG, Kaohsiung City (TW); Chia-Kuang TSUNG, Jhongli City (TW)

(21) Appl. No.: 15/070,600

(22) Filed: Mar. 15, 2016

(30) Foreign Application Priority Data
Sep. 14, 2015 (TW) 104130279

(51) Int. Cl. C12N 11/04; C07F 3/06; C12N 9/08

(52) U.S. Cl. CPC C12N 11/04; C12N 9/0065; C12Y 111/01006; C07F 3/06

(57) ABSTRACT

The invention provides a molecule carrier, comprising a metal-organic framework having an interior space and a surface of the metal-organic framework has a plurality of pores; and a molecule embedded in the interior space of the metal-organic framework. The invention also provides a method for preparing the molecule carrier by a de novo approach, comprising mixing a solution containing metal ions, an organic ligand, a molecule, and a surface coating agent to form an aqueous mixture. After incubating for a few minutes, the aqueous mixture is subjected to a drying process to obtain the molecule carrier.

### CROSS REFERENCE TO RELATED APPLICATION

This application claims the benefits of the Taiwan Patent Application Serial Number 104130279, filed on Sep. 14, 2015, the subject matter of which is incorporated herein by reference.

### BACKGROUND OF THE INVENTION

1. Field of the Invention

The invention provides a molecule carrier and a method for preparing the same. In particular, a molecule is embedded into the internal space of the metal-organic framework of the molecule carrier rather than adsorbed onto the metal-organic framework. The invention also provides a method for rapidly preparing the molecule carrier via a quick and one-step de Novo approach.

2. Description of Related Art

Since biocatalysts promote various chemical reactions; thus, the size of global enzyme market is estimated to be about $1 billion. According to current researches, biological reagents are expected to be applied in areas including chemical industry, food industry (beverage production, meat preservation, enzymes for starch hydrolysis), pharmaceutical industry, petrochemical industry, environmental protection (such as polluted water treatment), and other areas. In recent years, a German research team has discovered a specific enzyme that can be applied to biological hydrogen storage systems. Such enzyme is expected to solve the existing problems of storage and transport of fuel cells.

However, biocatalysts are usually not stable enough in reaction so that biocatalysts being used for industrial applications are limited so far. As a result, one of the main tasks now is to develop biocatalysts having a potential in industrial application. Since one focus is on methods for improving the stability and the catalytic efficiency of biocatalysts, researchers have begun to study how to properly fix biological molecules, such as cells, microorganisms, or enzymes, onto a solid carrier.

Conventional solid carriers for fixing biological molecules include microparticles, hydrogels, and nanoporous inorganic materials (such as zeolites and molecular sieves). Researches have suggested that when an enzyme is immobilized on a carrier, not only the enzyme will have improved stability and catalytic efficiency, but the enzyme can also be recycled and re-used easily. Hence, the production costs can be reduced.

In previous studies, such biomolecules (such as enzymes) can be immobilized into a carrier or can also be immobilized on a surface of a carrier. If the biomolecule is designed to immobilize in a carrier, the biomolecule needs to enter into the interior space of the carrier. The pore size of the carrier must be big enough to allow the biomolecule to enter. The binding force between the biomolecule and the carrier needs to be strong enough to prevent the biomolecule from leaching out of the carrier. On the other hand, if the biomolecule is designed to adsorb on a surface of a carrier, the exposed biomolecule may interfere with other factors existed in the external environment, thereby reducing the effect of the biomolecule.

Furthermore, since additional designs (such as functional group modification, etc.) are required to ensure that the biomolecule is properly bound to the carrier, the preparation process is often time-consuming. If the biomolecule (such as enzyme) has a conformational change due to any environmental factors of synthesis (such as temperature or pH value) during the immobilization process, the biological activity of the biomolecule will be degraded and the effect of the biomolecule will not be as perfect as expected.

Recent studies have proposed a metal organic framework (MOF) as a carrier. The so-called “metal organic framework (MOF)” is a highly crystalline compound complex composed of certain materials and usually has a frame structure. Through different coordination, bonding, and combinations of metals and organic molecules, metal-organic frameworks (MOF) with different specific properties can be prepared. The porosity of metal organic framework (MOF) gives metal organic framework (MOF) a wider application. For example, metal organic framework (MOF) can be used in areas such as gas storage, sensors, adsorption, separation, or catalysis technology.

However, when metal organic framework (MOF) is used as a carrier, the biomolecule, such as enzyme, which is a large size molecule, often has difficulty entering into the metal-organic framework (MOF). As a result, previous studies have often synthesized metal-organic framework (MOF) first followed by physically adsorbing or covalently bonding the biomolecule to the pre-synthesized carrier. As described previously, since the biomolecule is adsorbed to the outside of metal-organic framework (MOF), the biomolecule can easily interact with other factors existed in the external environment. Consequently, the function of the biomolecule may be affected, resulting in decreased function or even function being lost. More specifically, if the environment contains proteolytic enzymes or chemical molecules that inhibit enzyme, the activity of the biomolecule adsorbed on metal-organic framework (MOF) may be degraded or inhibited.

Therefore, there is a need to provide a novel molecule carrier and a simple method for preparing the molecule carrier that can overcome the above drawbacks in order to widen the application of the molecule carrier.

### SUMMARY OF THE INVENTION

The object of the invention is to provide a novel molecule carrier, in which a metal-organic framework (MOF) is used as a carrier for molecule immobilization. More specifically, in the molecule carrier of the invention, molecules are embedded into the metal-organic framework (MOF). Since different metal-organic frameworks (MOF) have different pore shapes and sizes, which is absent in porous materials such as zeolites and other inorganic porous materials, metal-organic frameworks (MOF) is more suitable to be used to combine with a wide variety of biomolecules.

The molecule carrier of the invention not only is reusable, but the pores of the metal-organic framework (MOF) are also a size-selective shelter. Thus, destructive molecules (such as proteolytic enzymes, inhibitors, etc.) are kept out of the metal-organic framework (MOF) so that the biomolecules within the metal-organic framework (MOF) are able to function properly without being affected by the destructive molecules. Furthermore, biomolecules with sizes smaller than the pores of the metal-organic framework (MOF) are kept in the metal-organic framework (MOF) so that the leaching of these biomolecules out of the metal-organic framework (MOF) is prevented.

Specifically, the molecule carrier of the invention comprises: a metal-organic framework having an interior space and a surface of the metal-organic framework has a plurality of pores; and a molecule embedded in the interior space of the metal-organic framework. In the invention, the molecule is embedded into the interior space of the metal-organic framework (MOF) and bound to the metal-organic framework (MOF) directly. The molecule is completely stored in the interior space of the metal-organic framework (MOF).

The surface of the metal-organic framework (MOF) has pores. A diameter of the pores is in a range from 1 Å to 2 nm and preferably from 2 Å to 1 nm, but is not limited thereto. The preferred diameter of the pores of the molecule carrier can be changed based on different needs. For example, in an example of the invention, since the molecule is a catalase, the preferred diameter of the pores may be 3.5 Å.

In the invention, a diameter of the pores of the metal-organic framework (MOF) may be smaller than a size of the molecule to prevent the molecule from leaching out of the pores.

In the invention, the metal-organic framework (MOF) may be a transition metal-based metal-organic framework (MOF). For example, the metal-organic framework (MOF) may be a zinc-based metal-organic framework (MOF), a cobalt-based metal-organic framework (MOF), a zirconium-based metal-organic framework (MOF), a chromium-based metal-organic framework (MOF), or other transition metal-based metal-organic frameworks (MOF). More specifically, the metal-organic framework (MOF) may be ZIF-8, ZIF-67, ZIF-90, or the likes. In an example of the invention, a zinc-based metal-organic framework (MOF), ZIF-90, is used. However, the invention is not limited thereto and a person having ordinary skills in the arts can select a proper metal-organic framework (MOF) according to the molecular size of the molecule to be encapsulated.

In the invention, the molecule of the invention may be a biological molecule or a non-biological molecule. When biological molecules are used, DNA, RNA, a protein (such as enzymes), or the combination thereof can be used. When non-biological molecules are used, a small molecule drug or an inhibitor can be used. In an example of the invention, the molecule can be an enzyme, such as a catalase; however, the invention is not limited thereto. The molecule can also be other enzymes. The molecule carrier can also be used to encapsulate molecules other than enzymes. For example, if the molecule is a drug, the molecule carrier of the invention can be used as a pharmaceutical product with sustained release.

The invention also provides a convenient and time-saving method for preparing the said molecule carrier. Specifically, the molecule carrier is prepared through a one-step de novo process in which the molecule is being embedded into the metal-organic framework (MOF). In the molecule carrier of the invention, even though the diameter of the pores is smaller than the size of the molecule, the molecule can still be embedded into the interior space of the metal-organic framework (MOF) in an easy manner. Hence, the molecule carrier of the invention can be widely applied in different industries.

More specifically, in the invention, a method for preparing a molecule carrier comprises: mixing a solution containing metal ions, an organic ligand, a molecule, and a surface coating agent to form an aqueous mixture, and then drying the aqueous mixture to obtain the molecule carrier of the invention. During the preparation, if necessary, the aqueous mixture may be rinsed by deionized water (DI water) before drying.

The method of the invention may be performed at a temperature of 4° C. to 60° C., where an organism can survive. For example, the preferred temperature in the examples of the invention is from 35° C. to 45° C. However, the method of the invention may also be performed at room temperature as well. Herein, the “room temperature” may be in a range from 15° C. to 35° C.

Furthermore, the solution containing metal ions used in the invention is not particularly limited and may be different depending on the type of the metal-organic framework (MOF) to be prepared. The solution containing metal ions may be a solution containing transition metal ions, such as a solution containing a metal salt of zinc, a solution containing a metal salt of cobalt, a solution containing a metal salt of zirconium, or a solution containing a metal salt of chromium. In an example of the invention, a solution containing a metal salt of zinc is used to prepare a zinc-based metal-organic framework (MOF).

In the invention, the metal-organic framework (MOF) is formed through self-assembling and connecting the inorganic metal centers and the organic ligands bridged to the inorganic metal centers. Different organic ligands may be used according to different needs, such as imidazole-2-carboxaldehyde, 2-methyl imidazole, imidazole derivatives, or terephthalic acid and derivatives thereof. The organic ligand connects to the inorganic metal of the aforesaid solution containing metal ions. For example, in an example of the invention, the organic ligand is imidazole-2-carboxaldehyde. However, the organic ligand used in the invention is not limited thereto.

In the method for preparing a molecule carrier of the invention, a molecule can be embedded in the metal-organic framework (MOF) directly during the synthesis of the metal-organic framework (MOF). The molecule may be DNA, RNA, a protein, a drug, an inhibitor, the likes, or the combination thereof. However, the type of molecule to embed is not particularly limited and can be different according to different uses. In an example of the invention, the molecule can be an enzyme.

Therefore, the invention provides a method, which can rapidly prepare the molecule carrier via a de Novo approach. The method provided can be performed at a temperature where a living organism can survive and can even be performed at room temperature. The molecule carrier can also be synthesized in an aqueous phase. The aforesaid mild reaction condition protects the encapsulated biomolecule from being damage. In the obtained molecule carrier, since a molecule is embedded into the metal-organic framework (MOF), the leaching of the molecule out of the metal-organic framework (MOF) is prevented. The pore size of the metal-organic framework (MOF) can be adjusted based on the substrate size of an enzyme. The pores of the metal-organic framework (MOF) also provide a size selective shelter to protect the molecule within the metal-organic framework (MOF) from being affected or destroyed by environmental factors.

Other objects, advantages, and novel features of the invention will become more apparent from the following detailed description when taken in conjunction with the accompanying drawings.

### BRIEF DESCRIPTION OF THE DRAWINGS

FIG. 1 is a schematic view of a structure of a preferred example of a molecule carrier of the invention;

FIGS. 2A and 2B are respectively a scanning electron microscope (SEM) image and an X-ray diffraction (XRD) pattern of a preferred example of a molecule carrier of the invention;

FIGS. 3A and 3B are respectively a nitrogen sorption isotherm and a TAG curve of a preferred example of a molecule carrier of the invention;

FIGS. 4A and 4B show that catalase and myoglobin are embedded into the metal-organic framework ZIF-90 of a molecule carrier of the invention; and

FIG. 5 shows a kinetic of H₂O₂ degradation by a preferred example of a molecule carrier of the invention.

### DETAILED DESCRIPTION OF THE PREFERRED EMBODIMENT

In order for a person skilled in the art to understand the purposes, technical features, and advantages of the invention, the following descriptions will be described in detail with drawings and preferred embodiments of the invention.

### Example 1

Zinc nitrate (371.3 mg) was added to deionized water (3.0 mL) to form an aqueous zinc nitrate solution at room temperature of 20° C. to 30° C.

Meanwhile, at 42° C., imidazole-2-carbaldehyde (ICA, 480.0 mg), polyvinylpyrrolidone (PVP, 50.0 mg), and catalase extracted from bovine liver (25.0 mg) were dissolved in deionized water (25.0 mL) to form an mixture. The mixture was then mixed with the said aqueous zinc nitrate solution to form an aqueous mixture. The aqueous mixture was stirred for about 10 minutes.

Subsequently, the said aqueous mixture was subjected to centrifugation at 14,000 g. The obtained product was rinsed by deionized water followed by drying in vacuum at room temperature to obtain a molecule carrier (hereinafter, referring as “CAT@ZIF-90”).

### Example 2

Fluorescently labeled-catalase molecule (FITC-CAT) was synthesized and replaced the catalase of Example 1. The molecule carrier of Example 2 was synthesized in a manner same as Example 1 to obtain a molecule carrier (hereinafter, referring as “FITC-CAT@ZIF-90”).

### Comparative Example 1

Except for not adding the catalase extracted from bovine liver, the preparation steps were the same as described in Example 1 to obtain a molecule carrier (hereinafter, referring as “ZIF-90”).

### Comparative Example 2

The “ZIF-90” of Comparative Example 1 and the catalase of Example 1 were physically mixed by stirring to obtain a molecule carrier. In the molecule carrier of Comparative Example 2, the catalase adsorbed only on the outer surface of the ZIF-90 (hereinafter, referring as “CAT-on-ZIF-90”).

### Comparative Example 3

The “ZIF-90” of Comparative example 1 and the fluorescently labeled-catalase molecule (FITC-CAT) were physically mixed by stirring to obtain a molecule carrier. In the molecule carrier of Comparative Example 3, the fluorescently labeled-catalase molecule (FITC-CAT) adsorbed only on the outer surface of the ZIF-90 (hereinafter, referring as “FITC-CAT-on-ZIF-90”).

### Experimental Example 1

### Structure of CAT@ZIF-90

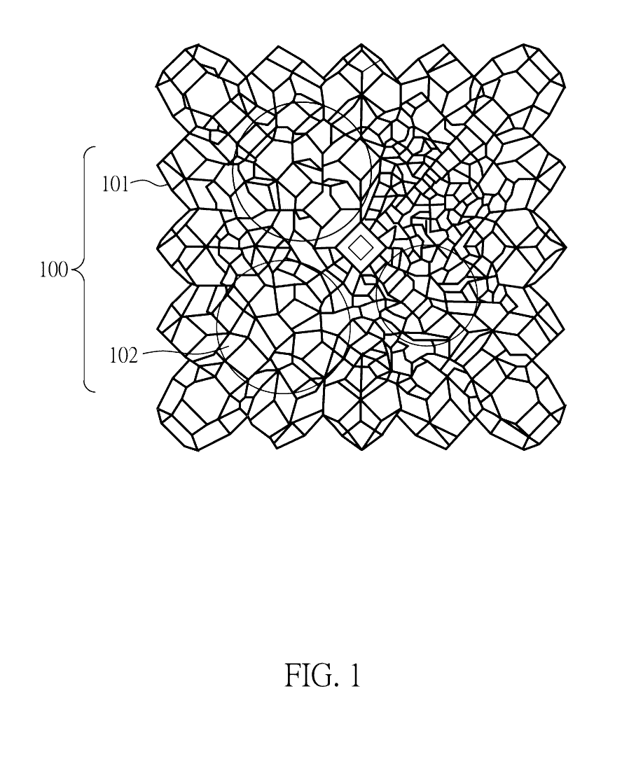

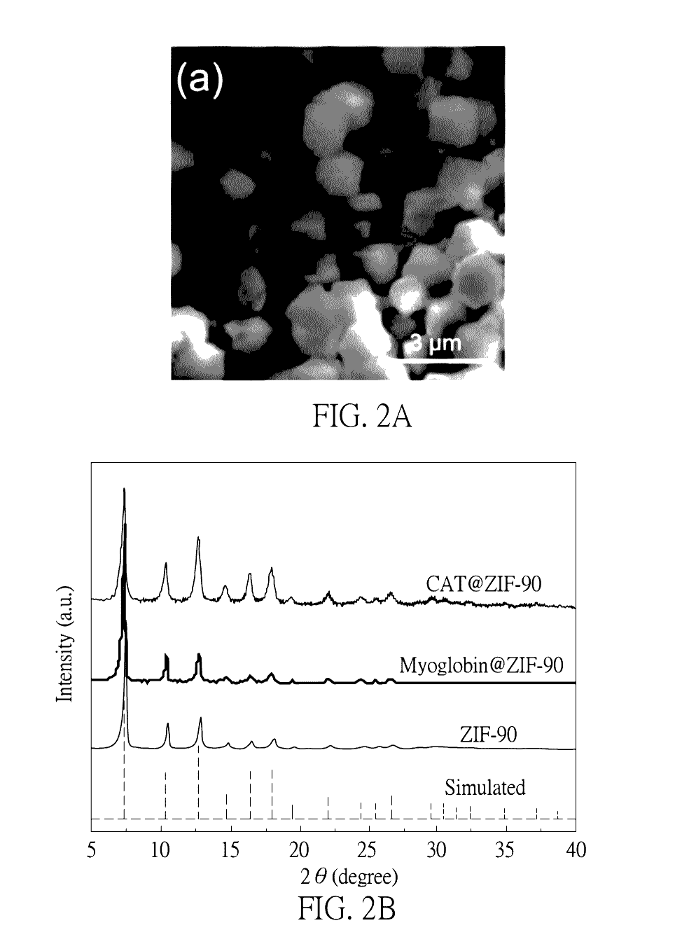

FIG. 1 is a schematic view of a structure of a molecule carrier 100 of the invention. The molecule carrier 100 comprises a metal-organic framework (MOF) 101 (ZIF-90). The metal-organic framework (MOF) 101 has an interior space and a surface of the metal-organic framework (MOF) 101 also has a plurality of pores. Molecule 102 (catalase) is embedded in the interior space of the metal-organic framework (MOF) 101. The structure of CAT@ZIF-90 of Example 1 was observed using a scanning electron microscope (SEM), which is shown in FIG. 2A. As shown, CAT@ZIF-90 has a uniform size of about 1-2 μm. According to the X-ray diffraction (XRD) patterns shown in FIG. 2B, there were no significant differences in the crystal structures and degrees of crystallinity of CAT@ZIF-90 and ZIF-90.

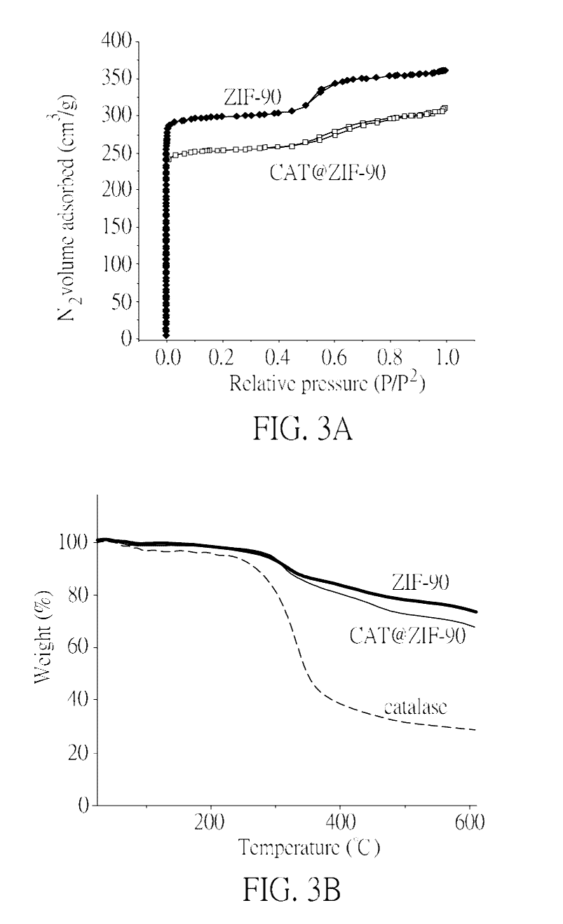

Next, the porous features of CAT@ZIF-90 and ZIF-90 were investigated using nitrogen sorption isotherms obtained by Micromeritics ASAP 2010 analyzer. As shown in FIG. 3A, the results obtained were similar to typical adsorption isotherm Type I (also known as Langmuir type). Therefore, CAT@ZIF-90 and ZIF-90 can be deemed to have microporous structures.

In addition, specific surface areas calculated by Langmuir and BET adsorption-desorption isotherm models are shown in Table 1. Since the catalase was embedded into the porous material; thus, the catalase occupied part of the surface area of the porous material. As a result, as shown in Table 1, CAT@ZIF-90 has smaller Langmuir surface area (S_{L}), BET surface area (S_{BET}), and total pore volume than ZIF-90.

TABLE 1

| | | | | t-plot |
|---|---|---|---|---|
| | Langmuir | | total | micropore |
| | surface | | pore | volume: |
| | area: S_{L} | S_{BET} | volume | V_{micro} |
| | (m²/g) | (m²/g) | (cm³/g) | (cm³/g) |
| Comparative example 1: | 1309 | 992 | 0.55 | 0.38 |
| ZIF-90 | | | | |
| Example 1: CAT@ZIF-90 | 1111 | 843 | 0.47 | 0.37 |

FIG. 3B shows a result of Thermogravimetric Analysis (TGA), which measures weight change of a sample under a specific temperature condition to provide information of weight loss with respect to the temperature of a sample. As shown, at about 320° C., CAT@ZIF-90 had a slight weight loss compared to ZIF-90. The pattern of CAT@ZIF-90 was similar to the catalase decomposition curve, indicating catalase decomposition in CAT@ZIF-90. In other words, the result confirmed the existence of catalase in CAT@ZIF-90.

### Experimental Example 2

Catalase was indeed embedded in the metal-organic framework (MOF) ZIF-90.

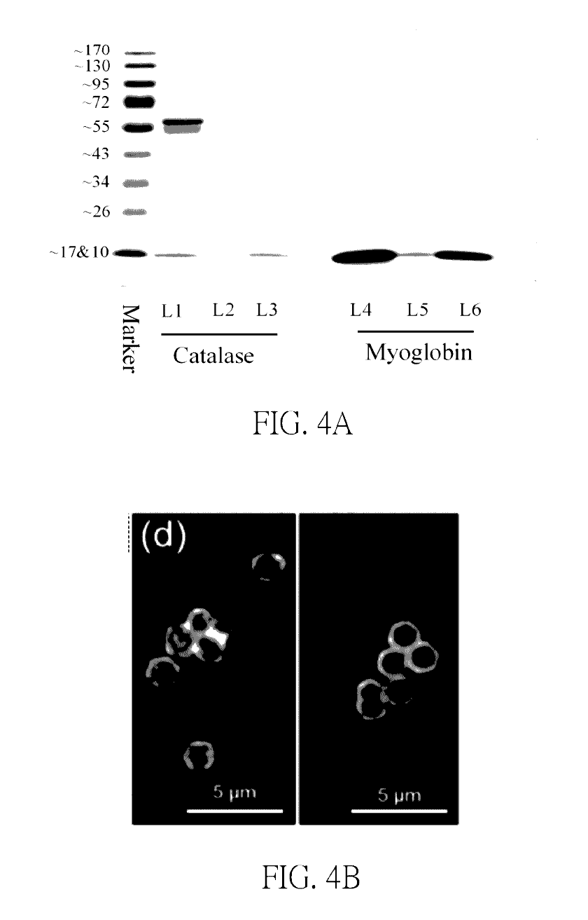

Experimental Example 2 confirmed that the catalase was indeed embedded in the metal-organic framework (MOF) ZIF-90 instead of being absorbed on the external surface of the metal-organic framework (MOF) ZIF-90. In Experimental Example 2, after rinsing CAT@ZIF-90 of Example 1 and CAT-on-ZIF-90 of Comparative Example 2 by deionized water, an acid was used to dissolve the metal-organic framework (MOF) material to release the molecule (protein). The molecule (protein) was then analyzed by sodium dodecyl sulfate polyacrylamide gel electrophoresis (SDS-PAGE). As shown in FIG. 4A, column 1 was the catalase of Example 1 (“L1”), column 2 was CAT-on-ZIF-90 of Comparative Example 2 (“L2”), and column 3 was CAT@ZIF-90 of Example 1 (“L3”). Proteins with a molecular weight of about 60 KDa were detected in “L1” and “L3”, which corresponded to a single molecular weight of the catalase of Example 1. However, such protein was not detected in “L2”. The result suggested that the catalase was embedded into ZIF-90 of Example 1; thus, the catalase could not be washed away easily by deionized water. On the contrary, in Comparative Example 2, the catalase was adsorbed on the surface of ZIF-90; thus, the catalase could be washed away easily by deionized water.

Another experiment was conducted to prove that the catalase was embedded into the metal-organic framework (MOF) ZIF-90. Specifically, FITC-CAT@ZIF-90 of Example 2 and FITC-CAT-on-ZIF-90 of Comparative Example 3 were observed using a confocal microscope. The results are shown in FIG. 4B. (Note that in order to protect the catalase from being washed away, the rinsing step of the molecule carrier of Comparative Example 3 was omitted). As shown in FIG. 4B, the fluorescence of FITC-CAT@ZIF-90 of Example 2 had a more uniform distribution (FIG. 4B, left) compared to the fluorescence of FITC-CAT-on-ZIF-90 of Comparative Example 3, which distributed only at the edge (FIG. 4B, right). The result indicated that the catalase was embedded in the metal-organic framework (MOF) in Example 2 rather than being adsorbed onto a surface of the metal-organic framework (MOF) in Comparative Example 3.

### Experimental Example 3

The preparation method of the invention retained the biological activity of the molecule.

It is known that catalase can decompose hydrogen peroxide to water and oxygen. In Experimental Example 3, degradation kinetics of hydrogen peroxide were studied to evaluate the biological activity of the catalases embedded into a metal-organic framework (MOF) prepared via an aqueous phase-preparation method (present invention) and a conventional alcohol phase-preparation method.

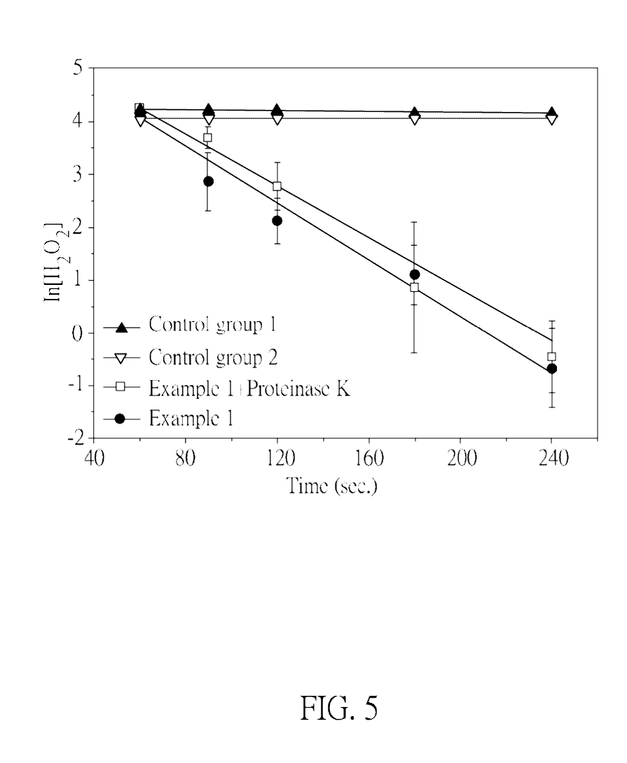

FIG. 5 shows result of FOX assay. During FOX assay, iron divalent ions (Fe²⁺) of the FOX reagent will react with the remaining hydrogen peroxide and become iron trivalent ions (Fe³⁺). The said iron trivalent ions (Fe³⁺) and xylenol orange will form complexes under slightly acidic condition. A good linear absorption intensity at UV-Vis 560 nm will be obtained. Thereby, a concentration of hydrogen peroxide will be obtained indirectly. As shown in FIG. 5, CAT@ZIF-90 of Example 1 was measured to have an observed rate constant (k_{obs}) of 0.0268 S⁻¹. However, the biological activity of the catalase immobilized on the molecule carrier obtained by the conventional alcohol phase-preparation method (referring as “control group 1”; the preparation method was the same as Example 1 except alcohol was used as the solvent instead of water) could not be detected. This was possibly due the destruction of the catalase by the organic solvent (alcohol).

It is also known that enzyme (catalase) activity might be affected by substances existed in the environment. For example, enzyme (catalase) activity may be weakened by substances in the environment. To prove the embedded catalase of CAT@ZIF-90 of Example 1 can be protected from substances in the environment, free catalase (referring as “control group 2”; the “free catalase” meant the catalase was not being bound to any carrier) and CAT@ZIF-90 of Example 1 were respectively mixed with proteinase K. It should be noted that proteinase K has a molecular size of 68.3×68.3×108.5 Å (28.5 kDa), which is greater than the pore size of CAT@ZIF-90 of Example 1. As shown in FIG. 5, the free catalase was inhibited by protease K and its enzyme activity was lost in control group 2 (control group 2: catalase+protease K). In contrast, the enzyme activity of the catalase molecule of CAT@ZIF-90 was maintained (k_{obs}=0.0246 S⁻¹) (Example 1+protease K).

In addition to the aforesaid catalase as the molecule embedded into the metal-organic framework (MOF) ZIF-90, another example of the invention used myoglobin as the molecule to be embedded into the metal-organic framework (MOF) ZIF-90, named as Myoglobin@ZIF-90. The preparation method of Myoglobin@ZIF-90 was the same as that of the Example 1, except that myoglobin was used to replace the catalase of Example 1.

As shown in FIG. 2B, there were no significant differences in the crystal structures and degrees of crystallinity between Myoglobin@ZIF-90 and CAT@ZIF-90 and ZIF-90.

Moreover, following a method similar to Comparative Example 2, myoglobin was mixed with ZIF-90 and myoglobin was adsorbed onto the external surface of ZIF-90, named as Myoglobin-on-ZIF-90. Furthermore, following the same manner as Experimental Example 2, the sodium dodecyl sulfate polyacrylamide gel electrophoresis (SDS-PAGE) results indicated that myoglobin was indeed embedded into ZIF-90 rather than being adsorbed onto the external surface of ZIF-90. Specifically, please refer to FIG. 4, “L4” represented myoglobin, “L5” represented Myoglobin-on-ZIF-90, and “L6” represented Myoglobin@ZIF-90. As shown in FIG. 4, myoglobin was detected in “L4” and “L6” but was not detected in “L5”. This result suggested that myoglobin was embedded into ZIF-90 so that myoglobin could not be washed away easily by deionized water.

Although the invention has been explained in relation to its preferred embodiments, it is to be understood that many other possible modifications and variations can be made without departing from the spirit and scope of the invention as hereinafter claimed.

What is claimed is:

1. A molecule carrier, comprising:
a metal-organic framework having an interior space and a surface of the metal-organic framework has a plurality of pores; and
a molecule embedded in the interior space of the metal-organic framework.

2. The molecule carrier as claimed in claim 1, wherein a diameter of the pores is smaller than a size of the molecule.

3. The molecule carrier as claimed in claim 1, wherein the metal-organic framework is a transition metal-based metal-organic framework.

4. The molecule carrier as claimed in claim 1, wherein the molecule is DNA, RNA, a protein, a drug, an inhibitor, or the combination thereof.

5. The molecule carrier as claimed in claim 1, wherein the molecule is an enzyme.

6. A method for preparing a molecule carrier, comprising: mixing a solution containing metal ions, an organic ligand, a molecule, and a surface coating agent to form an aqueous mixture, and then drying the aqueous mixture.

7. The method as claimed in claim 6, wherein the method is performed at 4° C. to 60° C.

8. The method as claimed in claim 6, wherein the solution containing metal ions is a solution containing transition metal ions.

9. The method as claimed in claim 6, wherein the organic ligand is imidazole-2-carboxaldehyde, 2-methyl imidazole, imidazole derivatives, or terephthalic acid and derivatives thereof.

10. The method as claimed in claim 6, wherein the molecule is DNA, RNA, a protein, a drug, or an inhibitor.
